(19) United States
(12) Patent Application Publication
(10) Pub. No.: US 2008/0218686 A1
(43) Pub. Date: Sep. 11, 2008

(54) LENS CARE SOLUTIONS FOR USE WITH CONTACT LENSES OR CONTACT LENS CASES THAT CONTAIN SILVER

(76) Inventors: Ravi Sharma, Fairport, NY (US); Erning Xia, Penfield, NY (US)

Correspondence Address: Bausch & Lomb Incorporated, One Bausch & Lomb Place, Rochester, NY, 14604-2701, US

(21) Appl. No.: 12/038,014

(22) Filed: Feb. 27, 2008

Related U.S. Application Data
(60) Provisional application No. 60/893,639, filed on Mar. 8, 2007.

(51) Int. Cl. A61L 12/08; B08B 3/08; G02C 7/04

(52) U.S. Cl. 351/160 R; 134/42; 422/28

(57) ABSTRACT

Contact lens care solutions that are formulated for disinfecting or packaging contact lenses that comprise silver, or for disinfecting or storing contact lenses in contact lens cases that comprise silver.

This application claims the benefit of Provisional Patent Application No. 60/1893,639 filed Mar. 8, 2007 which is incorporated herein by reference.

The invention is directed to contact lens care solutions that are formulated for contact lenses that contain silver, or for contact lenses that are disinfected or stored in contact lens cases that contain silver.

### BACKGROUND OF THE INVENTION

Contact lenses have been used commercially to improve vision since the 1950s. The first contact lenses were made of hard materials. Later developments gave rise to soft (hydrogel) contact lenses, which presently represent 90% or more of the contact lens market in the United States and Europe. The increase in comfort level and oxygen permeability of soft contact lenses allow patients to wear the lenses for extended periods. The use of extended-wear lenses, however, can present an opportunity for the buildup of ocular proteins, bacteria or other microbes on the surfaces of the lenses. The build-up of these types of contaminents is not unique to soft contact lenses and can also occur with the use of hard contact lenses.

U.S. patent application Ser. No. 10/028,400 describes soft contact lenses containing silver and a polymer that is said to reversibly complex the silver to the lens polymer matrix. The patent application also describes incorporating silver into a lens case. Again, the described monomers can be used to reversibly complex silver to the case polymer matrix. Storing lenses in such cases is said to inhibit the growth of bacteria on said lenses.

U.S. patent application Ser. No. 10/891,407 describes making contact lenses with specific chelating polymer that can reversibly bind silver nanoparticles. The described method includes preparing a polymerizable mixture comprising a siloxane-containing macromer, vinylic monomer and a silver salt. Prior to or during polymerization the silver salt is reduced, and one observes silver nanoparticles in the polymer matrix. It is reported that the silver nanoparticles distributed in the lens provide an effective antimicrobial capability over extended periods of time. It is also reported that the silver nanoparticles are released from the lens matrix at a rate that inhibits the growth of microorganisms.

PCT publication WO 02/38161 to Smith discloses polyhexamethylene biguanide (“PHMB”) disinfecting solutions containing less than 0.05%, by weight, sodium chloride. However, because the solutions still add considerable amounts of sodium chloride for tonicity and use hydrochloric acid for pH adjustment, the solutions still contain significant amounts of chloride ion.

### SUMMARY OF THE INVENTION

The invention is directed to contact lens care solutions that are formulated for disinfecting or packaging contact lenses that comprise silver, or for disinfecting or storing contact lenses in contact lens cases that comprise silver.

The invention is also directed to a sealed, contact lens package that includes an enclosure. The enclosure comprises a contact lens that contains silver and a contact lens solution comprising less than 0.05% w/v chloride ion.

### DETAILED DESCRIPTION OF THE INVENTION

The use of contact lenses require that patients periodically disinfect or clean their contact lenses to remove a variety of ocular contaminants including bacteria and other microbes that can accumulate on the lenses. The disinfection or cleaning process typically requires that the patient place a used lens in a lens case and then treat the contact lens with a lens solution. Patients are instructed to consistently care for their contact lenses according to prescribed guidelines provided by the lens solution or contact lens manufacturer as well as their doctor. Proper contact lens care is very important to ocular health and to avoid problems that can be associated with contact lens wear if these guidelines are not followed.

Most, if not all, contact lens solutions on the market today contain one or more antimicrobial components to kill and inhibit the growth of bacteria and other microbes, e.g., fungi, on the contact lens. The killing of these microbes is very important to maintaining the ocular health of patients who wear contact lenses. The most common antimicrobial components are what are referred to as cationic antimicrobial components, which are described in greater detail later in this application.

Unfortunately, some patients over time begin to take shortcuts in the maintenance and care of their contact lenses. For example, some patients may not fully discard the lens care solution following each use, but instead, simply refill or top-off the lens container with fresh solution. Other patients may even attempt to use the same lens care solution for multiple disinfection cycles. In such instances, the antimicrobial efficacy of the solution can decrease over time and result in an increasing population of bacteria or fungi on the lens. This can lead to a serious ocular infection.

To provide additional protection against bacteria and other microbes some contact lens manufacturers have developed contact lenses or contact lens cases that contain silver. Since ancient times silver has been known to function as an antimicrobial agent. See, Matsumura et al., Applied and Environmental Biology, July 2003, 4278-81. Accordingly, many anti-infective wound dressing are known to contain silver. One well known application of silver containing solutions has been the use of such solutions in the eyes of newborns.

Applicants have referred to at least two U.S. patent applications above that describe the preparation of soft contact lenses that contain silver. These same two U.S. patent applications also describe incorporating silver into a lens case. Storing lenses in such cases is said to inhibit the growth of bacteria on said lenses. Morover, PCT publication WO2000/038552 describes a lens case containing silver in which the silver is reported to be complexed in a zeolite that is incorporated into the lens polymer matrix.

Applicants have discovered that if one uses lens care solutions that contain significant amounts of chloride ion, that is, 0.05% w/v or greater of chloride ion, with contact lenses that contain silver, or with contact lens cases that contain silver, one observes the precipitation of AgCl over time in either the lens care package solution or in the lens care solution during a disinfection cycle. To avoid or minimize the precipitation of AgCl, applicants have developed formulations that contain little or no chloride ion.

The invention is directed to a method of disinfecting, cleaning or packaging a contact lens. The method includes providing a contact lens comprising silver and positioning the contact lens in a lens case or lens package. The positioned contact lens is contacted with a lens solution that comprises less than 0.05% w/v chloride ion. Preferably, the contact lens solution comprises less than 0.01% w/v chloride ion, and more preferably, the lens solution comprises less than 0.005% w/v chloride ion.

Alternatively, the method includes providing a contact lens and positioning the lens in a lens case that contains silver. Again, the positioned lens is contacted with a contact lens solution that comprises less than 0.05% w/v chloride ion. Preferably, the contact lens solution comprises less than 0.01% w/v chloride ion, and more preferably, the lens solution comprises less than 0.005% w/v chloride ion.

It is to be understood by one of ordinary skill in the art, that one can also use a contact lens that contains silver with a lens case that also contains silver.

The invention is also directed to a sealed contact lens package. The lens package comprises a contact lens that contains silver and a contact lens solution comprising less than 0.05% w/v chloride ion. Preferably, the contact lens solution comprises less than 0.01% w/v chloride ion, and more preferably, the lens solution comprises less than 0.005% w/v chloride ion.

Alternatively, the invention is directed to a contact lens case that comprises silver, and contains a contact lens and a contact lens solution comprising less than 0.05% w/v chloride ion. Preferably, the contact lens solution comprises less than 0.01% w/v chloride ion, and more preferably, the lens solution comprises less than 0.005% w/v chloride ion.

The contact lens care solutions described herein are formulated to be isotonic with the lachrymal fluid, and have a tonicity range from 200 mOsm/kg to 400 mOsmol/kg, which corresponds to a 0.8% w/v to 1.0% w/v chloride solution. Sodium chloride or potassium chloride has been traditionally used as tonicity agents in contact lens solutions. For example, a 0.9% (w/v) sodium chloride solution would result in almost 5500 ppm chloride ion in the solution. This is simply too much chloride in a solution that is used for packaging contact lenses that contain silver, or for disinfecting or storing contact lenses in contact lens cases that contain silver.

Accordingly, the solutions have less than 0.05% (w/v) chloride. More preferably, both sodium chloride and potassium chloride, if present at all, are present at less than 0.01% chloride (w/v), most preferably less than 0.005% chloride (w/v). In particular, the solutions have been formulated to include no sodium chloride or potassium chloride other than the presence of impurities that can exist in the other components of the formulation.

The term “contact lens solution” means a solution that is formulated as a “disinfecting solution” and thereby contains one or more antimicrobiocidal components, that is effective for reducing or substantially eliminating the presence of an array of microorganisms present on a contact lens. The term “contact lens solution” also means a solution that is formulated as a preserving solution, storage or packaging solution for contact lenses.

The term “silver” used herein includes silver present as elemental silver or complexed as in a chelating compound. The silver can be present in any one or more oxidation states, and includes silver nanoparticles. The term “silver nanoparticles” refers to particles made essentially of silver that have an average particle size of less than 0.1 microns. Silver nanoparticles contain silver in a primarily reduced form, that is, the Ag° oxidation state, though the Ag¹⁺ oxidation state can also coexist in the nanoparticles. The presence of silver nanoparticles in a contact lens or a polymer mixture used to make a lens case can be confirmed by UV spectroscopy with an absorption peak located in a wavelength range from about 390 nm to about 450 nm, which is a characteristic peak of silver nanoparticles.

An “ophthalmically safe” solution has a tonicity and pH that is compatible with the eye and comprises materials, and amounts thereof, that are non-cytotoxic according to international ISO standards and U.S. FDA regulations. It is preferred that the solutions of the present invention be ophthalmically safe.

The majority of the tonicity of the contact lens solution is provided by one or more compounds selected from the group consisting of non-halide containing electrolytes (e.g., sodium bicarbonate) and non-electrolytic compounds. By non-electrolyte, it is meant those compounds that do not readily dissociate into ions in water. Examples include, but are not limited to, sorbitol, mannitol, glycerol, propylene glycol, xylitol, and inositol. Such solutions have a tonicity in the range 200 to 450 mOsm/kg, preferably in the range 220 to 330 mOsm/kg, most preferably in the range of 270 to 310 mOsm/kg, and at least 50% of the tonicity of the solution will be provided by one or more non-halide containing electrolytes or non-electrolytic compounds. Specifically, the tonicity of the contact lens solution is provided by one or more non-electrolytic compounds which contribute to at least 60%, preferably at least 75%, and more preferably at least 85%, of the tonicity of the solution.

Organic compounds that can be used to adjust the tonicity of the contact lens solutions include, but are not limited to, glycerol, urea, propylene glycol, polyols, or carbohydrates and sugars such as mannitol or sorbitol, and inorganic substances such as sulfates and nitrates. Of course, the solutions can also include any one mixture comprised of any two or more of the above organic or inorganic compounds. In particular, the organic sugars such as mannitol or sorbitol.

The addition of sorbitol to adjust the tonicity of contact lens care products is known. GB 2,205,175 and U.S. Pat. No. 3,888,782 describe sorbitol as a carrier material for the preparation of powder mixtures for contact lens care products. Moreover, sorbitol is not cytotoxic and does not have negative effects on the antimicrobial efficacy of the solutions. Sorbitol is typically present in the contact lens solutions in an amount of 0.4% to 18 (w/v), from 2 (w/v) to 8% (w/v), or from 3% to 6% (w/v). Sorbitol can play an important role in establishing the necessary tonicity range of the solutions. Accordingly, the solutions will typically contain at least 3% sorbitol.

In solutions where at least 50% of the tonicity of the solution is provided by non-electrolytic compounds, the concentration of the biguanide anti-microbial compound can be greatly reduced. For example, the preferred anti-microbial compound, PHMB, can be employed effectively in a disinfecting solution against C. albicans at a concentration as low as 1 ppm, or even as low as 0.1 ppm. More preferably, PHMB is present less than or equal to 0.5 ppm.

Again, because the solutions described herein are used for treating soft contact lenses that contain silver nanoparticles, or are used for treating non-silver containing, soft contact lenses that are cleaned or stored in contact lens cases containing silver nanoparticles the solutions are virtually free of chloride ions. The solutions can, however, contain low amounts of phosphate ions the source of which is a phosphate buffer system. Solutions that contain less than 1000 ppm chloride ion and contain phosphate buffer at a concentration less than 0.1% can provide better cleaning abilities, while maintaining antimicrobial efficacy, as compared to solutions without phosphate buffers. Preferably, solutions that use a phosphate buffer will have less than 500 ppm chloride ion, less than 250 ppm chloride ion, or less than 100 ppm chloride ion. Furthermore, it is preferred that the total concentration of phosphate buffers (Na₂HPO₄, NaH₂PO₄, and KH₂PO₄, and mixtures thereof) is less than 0.06%, less than 0.025%, or between about 0.005% and 0.015%.

A solution that is useful for disinfecting, cleaning, storing, and rinsing a contact lens, is referred to herein as a “multi-purpose solution.” In most cases, the use of a multi-purpose solution does not require the patient to rinse the lens with a separate rinsing solution prior to placing the treated lens on the eye. Some patients however, particularly patients sensitive to chemical disinfectants or other chemical agents, may prefer to rinse or wet a contact lens with a another solution, e.g., a sterile saline solution prior to the placement of the lens. The term “multi-purpose solution” also does not exclude the use of periodic cleaners or supplemental cleaners for removing proteins, for example enzyme cleaners.

The cationic antimicrobial components include chemicals which derive their antimicrobial activity through a chemical or physiochemical interaction with microbes or microorganisms normally associated with contaminating a contact lens. Suitable antimicrobial components include, but are not limited to, quaternary ammonium salts used in ophthalmic applications such as poly[dimethylimino-2-butene-1,4-diyl] chloride, α-[4-tris(2-hydroxyethyl) ammonium chloride-2-butenyl]poly[1-dimethylammonium chloride-2-butenyl]-(ω-tris(2-hydroxyethyl)ammonium chloride (CAS#68518-54-7, available as Polyquaternium-1® from Onyx Corporation), myristamidopropyl dimethylamine (Aldox®), benzalkonium halides, and biguanides such as salts of alexidine, alexidine-free base, salts of chlorhexidine, hexamethylene biguanides and salts thereof and their polymers, antimicrobial polypeptides and mixtures thereof.

The term “cationic” when referring to an antimicrobial component refers to the predominant form of the antimicrobial component at neutral pH having a positive charge and a counter anion. An exemplary list of cationic disinfecting antimicrobial components include poly[dimethylimino-2-butene-1,4-diyl] chloride, α-[4-tris(2-hydroxyethyl) ammonium chloride-2-butenyl]poly[1-dimethylammonium chloride-2-butenyl]-ω-tris(2-hydroxyethyl)ammonium chloride, myristamidopropyl dimethylamine, alexidine, poly(hexamethylene biguanide) (PHMB), PHMB-CG* and any mixture thereof.

A new synthetic route to polymeric biguanide compositions is described in copending U.S. provisional application Ser. No. 60/853,579 filed Oct. 23, 2006, the entire disclosure of which is incorporated herein by reference. The new synthetic route provides a polymeric biguanide composition with relatively greater number of cyanoguanidino end groups. Typically, the number percent of cyanoguanidino end groups can be increased to greater than 60%. In this application we refer to this novel polymeric biguanide composition as PHMB-CG*. We also refer to polymeric biguanide compositions in the generic sense as “hexamethylene biguanides”, which one of ordinary skill in the art would recognize to include both PHMB as well as PHMB-CG*.

The cationic antimicrobial component is present in an amount from 0.01 ppm to 100 ppm, from 0.1 ppm to 50 ppm or from 0.1 ppm to 10 ppm. Typically, an amount of the antimicrobial component is used to reduce the microbial burden or load on the contact lens by one log order in four hours. More preferably, an effective amount of the antimicrobial component reduces the microbial load by one log order in one hour. The reductions are based upon similarly prepared lens solutions absent the cationic antimicrobial component.

In one embodiment, the primary antimicrobial component present in the lens care solutions is poly(hexamethylene biguanide) or PHMB-CG*, which is present from 0.01 ppm to 3 ppm. In another embodiment, the primary antimicrobial component present in the lens care solutions is α-[4-tris(2-hydroxyethyl)ammonium chloride-2-butenyl]poly[1-dimethylammonium chloride-2-butenyl]-ω-tris(2-hydroxyethyl) ammonium chloride, which is present from 1 ppm to 100 ppm.

Any one mixture of the two cationic antimicrobial components can also be present in the lens care solutions. For example, a particular lens care solution can include from 0.3 ppm to 0.8 ppm PHMB or PHMB-CG*, and 10 ppm to 60 ppm α-[4-tris(2-hydroxyethyl)ammonium chloride-2-butenyl]poly[1-dimethylammonium chloride-2-butenyl]-ω-tris(2-hydroxyethyl) ammonium chloride.

The method can also include adding a fatty acid monoester to a lens care solution The fatty acid monoester comprises an aliphatic fatty acid portion having ten carbon atoms, and an aliphatic hydroxyl portion. This lens care solution exhibits an enhanced efficacy against Candida albicans or Fusarium solani. The method has proven to be quite advantageous if the cationic disinfectant is poly(hexamethylene biguanide) or PHMB-CG*.

Alternatively, the fatty acid monoester comprises an aliphatic fatty acid portion having eight carbon atoms, and an aliphatic hydroxyl portion. This lens care formulation exhibits an enhanced efficacy against Staphylococcus aureus, pseudomonas aeruginosa or Serratia marcescens or any combination thereof. In both instances, the preferred aliphatic hydroxyl portion is based on glycerol.

The contact lens solutions in combination with a contact lens that contain silver or a contact lens case that contains silver are effective against a wide spectrum of microorganisms, including but not limited to Staphylococcus aureus (ATCC 6538), Pseudomonas aeruginosa (ATCC 9027), Serratia marcescens (ATCC 13880), Candida albicans (ATCC 10231), and Fusarium solani (ATCC 36031). Because C. albicans is typically the most difficult organism to kill, it is usually a good indicator of the overall effectiveness of a particular solution in combination with a lens or lens case that contains silver. The comparatively low levels of biguanides contemplated for use in the solutions are effective against a broad spectrum of microorganisms, including C. albicans, in a solution having less than 0.05% w/v of chloride ion.

The method can also include adding dexpanthenol, which is an alcohol of pantothenic acid, also called Provitamin B5, D-pantothenyl alcohol or D-panthenol, to the contact lens solution. In some formulations, dexpanthenol can exhibit good cleansing action and can stabilize the lachrymal film at the eye surface when placing a contact lens on the eye. Dexpanthenol is preferably present in the solutions in an amount from 0.01% to 5% (w/v), from 0.1% to 3% (w/v), or from 0.1% to 1% (w/v).

The method can also include adding one or more neutral or basic amino acids to the lens care solutions. The neutral amino acids include: the alkyl-group-containing amino acids such as alanine, isoleucine, valine, leucine and proline; hydroxyl-group-containing amino acids such as serine, threonine and 4-hydroxyproline; thio-group-containing amino acids such as cysteine, methionine and asparagine. Examples of the basic amino acid include lysine, histidine and arginine. The one or more neutral or basic amino acids are present in the solutions at a total concentration of from 0.1% to 5% (w/v).

The method can also include adding glycolic acid, aspartic acid or any mixture of the two at a total concentration of from 0.001% to 4% (w/v) or from 0.01% to 2.0% (w/v) to the lens care solutions. Further, the combined use of one or more amino acids and glycolic acid and/or aspartic acid can lead to a reduction in the change of the size of the contact lens due to swelling and shrinkage following placement of the lens on the eye. The stated combination provides a higher degree of compatibility with the contact lens compared to the absence of one of the two components in the composition. It is believed that one or more of the amino acids can cause the lens to swell, and that the glycolic acid and/or aspartic acid can cause the contact lens to shrink. If used in combination, however, a mutual counteraction of the two observed affects is believed to exist.

The method can also include adding glycolic acid, aspartic acid or any mixture of the two, in combination with 2-amino-2-methyl-1,3-propanediol or a salt thereof. One observed advantage is that solutions that do contain a mixture of two of the three, or all three, compounds minimizes the change of the lens size following placement of the contact lens in the eye. It is also believed that the stated combination of compounds minimizes the amount of uptake of the cationic antimicrobial component, particularly, α-[4-tris(2-hydroxyethyl) ammonium chloride-2-butenyl]poly[1-dimethylammonium chloride-2-butenyl]-ω-tris(2-hydroxyethyl) ammonium chloride, myristamidopropyl dimethylamine, benzalkonium halides, alexidine and salts thereof, salts of chlorhexidine, hexamethylene biguanides and salts thereof and their polymers such as poly(hexamethylene biguanide) or PHMB-CG*.

The 2-amino-2-methyl-1,3-propanediol (AMPD) or the salt thereof is added to the solutions in an amount to satisfy a predetermined molar ratio of glycolic acid, aspartic acid or any mixture of the two and AMPD. The molar ratio of the two components glycolic acid and/or aspartic acid to AMPD is 1:20 to 1.3:1. The glycolic acid, aspartic acid or any mixture of the two is present in the solutions at a concentration of 0.01% to 5% (w/v) or at a concentration of 0.05% to 1% (w/v).

If the components glycolic acid and/or aspartic acid, and AMPD, are present in the solutions in the absence of the other, one may observe a tendency to cause shrinkage or swelling of the lens. However, if these two components are combined together and used in the predetermined molar ratio, little, if any, change in the size of the lens is observed.

The amount of AMPD present in the solutions can be determined according to the amount of glycolic acid and/or aspartic acid in the composition. As stated, AMPD is present in an amount to provide a molar ratio of glycolic acid and/or aspartic acid to AMPD to be from 1:20 to 1.3:1, from 1:15 to 1.2:1 or from 1:14 to 1:1. If the amount of AMPD exceeds 20 mols per 1 mol of glycolic acid and/or aspartic, adsorption of the cationic antimicrobial component on the contact lens will occur. If the amount of AMPD is less than 1 mol per 1.3 mols of glycolic acid and/or aspartic acid, a reduction in antimicrobial efficacy of the solution is observed.

The contact lens care solutions will very likely include a buffer system. By the terms “buffer” or “buffer system” is meant a compound that, usually in combination with at least one other compound, provides a buffering system in solution that exhibits buffering capacity, that is, the capacity to neutralize, within limits, either acids or bases (alkali) with relatively little or no change in the original pH. Generally, the buffering components are present from 0.05% to 2.5% (w/v) or from 0.1% to 1.5% (w/v).

The term “buffering capacity” is defined to mean the millimoles (mM) of strong acid or base (or respectively, hydrogen or hydroxide ions) required to change the pH by one unit when added to one liter (a standard unit) of the buffer solution. The buffer capacity will depend on the type and concentration of the buffer components. The buffer capacity is measured from a starting pH of 6 to 8, preferably from 7.4 to 8.4.

Borate buffers include, for example, boric acid and its salts, for example, sodium borate or potassium borate. Borate buffers also include compounds such as potassium tetraborate or potassium metaborate that produce borie acid or its salt in solutions. Borate buffers are known for enhancing the efficacy of certain polymeric biguanides. For example, U.S. Pat. No. 4,758,595 to Ogunbiyi et al. describes that a contact-lens solution containing a polyaminopropyl biguanide (PAPB), also known asPHMB, can exhibit enhanced efficacy if combined with a borate buffer.

A phosphate buffer system preferably includes one or more monobasic phosphates, dibasic phosphates and the like. Particularly useful phosphate buffers are those selected from phosphate salts of alkali and/or alkaline earth metals. Examples of suitable phosphate buffers include one or more of sodium dibasic phosphate (Na₂HPO₄), sodium monobasic phosphate (NaH₂PO₄) and potassium monobasic phosphate (KH₂PO₄). The phosphate buffer components frequently are used in amounts from 0.01% or to 0.5% (w/v), calculated as phosphate ion.

Other known buffer compounds can optionally be added to the lens care solutions, for example, citrates, citric acid, sodium bicarbonate, TRIS, and the like. Other ingredients in the solution, while having other functions, may also affect the buffer capacity. For example, EDTA, often used as a complexing agent, can have a noticeable effect on the buffer capacity of a composition.

If a buffer such as TRIS is added to the solution at typical concentrations, the pH will be greater than 9.0 and must be adjusted downwardly. In the past it has been typical to employ hydrochloric acid to lower the pH. However, because the addition of hydrochloric acid would add unwanted chloride ions to the solution, it is preferred to either lower the concentration of TRIS to minimize the amount of acid needed to reach a suitable pH, or use acids that do not contain chloride ions. Of course, it is within the scope of this invention to utilize hydrochloric and to adjust pH downwardly, so long as the concentration of chloride does not exceed that range that define the solutions of the invention. Additionally, TRIS buffer is often supplied as TRIS.HCl, which again, would introduce additional chloride ion into the solution. Accordingly, it is preferred the “free-base” form of TRIS be used.

A preferred buffer system is based upon boric acid/borate, a mono and/or dibasic phosphate salt/phosphoric acid or a combined boric/phosphate buffer system. For example a combined boric/phosphate buffer system can be formulated from a mixture of sodium borate and phosphoric acid, or the combination of sodium borate and the monobasic phosphate.

In a combined boric/phosphate buffer system, the solution comprises about 0.05 to 2.5% (w/v) of a phosphoric acid or its salt and 0.1 to 5.0% (w/v)of boric acid or its salt. The phosphate buffer is used (in total) at a concentration of 0.004 to 0.2 M (Molar), preferably 0.04 to 0.1 M. The borate buffer (in total) is used at a concentration of 0.02 to 0.8 M, preferably 0.07 to 0.2 M.

Because the various phosphate buffers contribute differing amounts of phosphate ion to solution from the same percent by weight of buffer, the invention can alternatively be described in terms of ppm phosphate ion in the solution. In this manner, it is preferred that a phosphate buffered solution contain less than 800 ppm phosphate ion; especially less than 500 ppm phosphate ion, more preferably less than 200 ppm phosphate ion; most preferably between 40 ppm and 120 ppm phosphate ion. If less than about 0.055 ppm PHMB is present in the solution, it may be desirable to reduce the phosphate concentration even further.

The lens care solutions can also include a water-soluble borate-polyol complex which can be formed by mixing a source of borate with a polyol of choice in an aqueous solution. These complexes can be used in conjunction with the cationic antimicrobial component above, and can help to meet preservative efficacy and disinfection standards. In such solutions, the molar ratio of borate to polyol is generally from 1:0.1 to 1:10, or from 1:0.055 to 1:2.5. If present in the lens care solutions, the borate-polyol complex is usually present from 0.5% to 5% (w/v), from 1.0% to2.5% (w/v). The borate-polyol complexes are described in greater detail in U.S. Pat. No. 6,143,799.

The lens care solutions will very likely comprise effective amounts of one or more known lens care formulation components such as a detergent or surfactant component, a viscosity inducing or thickening component, a chelating or sequestering component, or a tonicity component. The additional component or components can be selected from materials which are known to be useful in contact lens care solutions and are included in amounts effective to provide the desired effect or benefit. When an additional component is included, it is preferably compatible under typical use and storage conditions with the other components of the solution.

Suitable surfactants can be either amphoteric, cationic, anionic, or nonionic, and are typically present (individually or in combination) in amounts up to 15%, or up to 5% (w/v). One preferred surfactant class are the amphoteric or nonionic surfactants. The surfactant should be soluble in the lens care solution and non-irritating to eye tissues. Many nonionic surfactants comprise one or more chains or polymeric components having oxyalkylene (—O—R—) repeats units wherein R has 2 to 6 carbon atoms. Preferred non-ionic surfactants comprise block polymers of two or more different kinds of oxyalkylene repeat units, which ratio of different repeat units determines the HLB of the surfactant. Satisfactory non-ionic surfactants include polyethylene glycol esters of fatty acids, e.g. coconut, polysorbate, polyoxyethylene or polyoxypropylene ethers of higher alkanes (C₁₂-C₁₈). Examples of the this class include polysorbate 20 (available under the trademark Tween® 20), polyoxyethylene (23) lauryl ether (Brij® 35), polyoxyethyene (40) stearate (Myrj®52), polyoxyethylene (25) propylene glycol stearate (Atlas® G 2612). Still other preferred surfactants include tyloxapol, betaine-type surfactants, polysulfates, polyethylene glycol, alkyl esters and any mixture thereof.

The solutions preferably contain tyloxapol. Tyloxapol is an oxyethylated tertiary octylphenol formaldehyde polymer, commercially available from Rohm & Haas Co. (Philadelphia, Pa.). Tyloxapol is a nonionic surfactant with surface tension reducing properties that is freely soluble in water. The tyloxapol can be used in concentrations ranging from 0.005% to 1.0% (w/v.), 0.01% to 0.5% (w/v), or 0.01% to 0.03% (w/v).

A particualr non-ionic surfactant consisting of a poly(oxypropylene)-poly(oxyethylene) adduct of ethylene diamine having a molecular weight from about 7,500 to about 27,000 wherein at least 40 weight percent of said adduct is poly(oxyethylene) has been found to be particularly advantageous for use in cleaning and conditioning both soft and hard contact lenses when used in amounts from about 0.01 to about 15 weight percent. The CTFA Cosmetic Ingredient Dictionary's adopted name for this group of surfactants is poloxamine. Such surfactants are available from BASF Wyandotte Corp., Wyandotte, Mich., under Tetronic®.

An analogous of series of surfactants, for use in the lens care compositions, is the poloxamer series which is a poly(oxyethylene) poly(oxypropylene) block polymers available under Pluronic® (commercially available form BASF). In accordance with one embodiment of a lens care composition the poly(oxyethylene)-poly(oxypropylene) block copolymers will have molecular weights from 2500 to 13,000 daltons or from 6000 to about 12,000 daltons. Specific examples of surfactants which are satisfactory include: poloxamer 108, poloxamer 188, poloxamer 237, poloxamer 238, poloxamer 288 and poloxamer 407. Particularly good results are obtained with poloxamer 237.

Various other ionic as well as amphoteric and anionic surfactants suitable for in the invention can be readily ascertained, in view of the foregoing description, from McCutcheon's Detergents and Emulsifiers, North American Edition, McCutcheon Division, MC Publishing Co., Glen Rock, N.J. 07452 and the CTFA International Cosmetic Ingredient Handbook, Published by The Cosmetic, Toiletry, and Fragrance Association, Washington, D.C.

Amphoteric surfactants suitable for use in a lens solution include materials of the type are offered commercially under the trade name “Miranol.” Another useful class of amphoteric surfactants is exemplified by cocoamidopropyl betaine, commercially available from various sources.

The foregoing surfactants will generally be present in a total amount from 0.01% to 5% (w/v), from 0.1% to 5% (w/v), or from 0.1% to 1.5% (w/v). Often the amount of surfactant is from 0.005% or 0.01%, to 0.1% or 0.5% or 0.8% (w/v).

The viscosity inducing components can also be added to the lens care solutions. Such viscosity inducing components are effective to enhance and/or prolong the cleaning and wetting activity of the surfactant component and/or condition the lens surface rendering it more hydrophilic (less lipophilic) and/or to act as a demulcent on the eye. Increasing the solution viscosity provides a film on the lens which may facilitate comfortable wearing of the contact lens. The viscosity inducing component can also function to cushion the impact on the eye surface during placement of the lens and serves also to alleviate eye irritation.

Suitable viscosity inducing components include, but are not limited to, water soluble natural gums, cellulose-derived polymers and the like. Useful natural gums include guar gum, gum tragacanth and the like. Useful cellulose-derived viscosity inducing components include cellulose-derived polymers, such as hydroxypropyl cellulose, hydroxypropylmethyl cellulose, carboxymethyl cellulose, methyl cellulose, hydroxyethyl cellulose and the like. A very useful viscosity inducing component is hydroxypropylmethyl cellulose (HPMC).

The viscosity inducing component is used in an amount effective to increase the viscosity of the solution, preferably to a viscosity in the range of about 1.5 to about 30, or even as high as about 750, cps at 25° C., as determined by USP test method No. 911 (USP 23, 1995).

A chelating or sequestering can be included in an amount effective to enhance the effectiveness of the cationic antimicrobial component and/or to complex with metal ions to provide more effective cleaning of the contact lens. A wide range of organic acids, amines or compounds which include an acid group and an amine function are capable of acting as chelating components in the present compositions. For example, nitrilotriacetic acid, diethylenetriaminepentacetic acid, hydroxyethylethylene-diaminetriacetic acid, 1,2-diaminocyclohexane tetraacetic acid, hydroxyethylaminodiacetic acid, ethylenediamine-tetraacetic acid and its salts, polyphosphates, citric acid and its salts, tartaric acid and its salts, and the like and mixtures thereof, are useful as chelating components.

In one embodiment, the lens care compositions include a phosphonic acid, or its physiologically compatible salt, as a chelating or sequestering agent. The phosphonic acid is represented by the following formula:

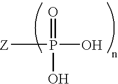

wherein Z is a connecting radical equal, n is an integer from 1 to 4, or 1, 2 or 3, and preferably containing 1 to 12 carbon atoms, more preferably 3 to 10 carbon atoms. The Z radical comprises substituted or unsubstituted saturated hydrocarbon radicals or amine-containing radicals, which amine-containing radicals are saturated hydrocarbon radicals in which the carbon atoms are interrupted with at least one nitrogen atom such as 1, 2 or 3 nitrogen atoms that forms a secondary or tertiary amine.

Accordingly, suitable Z radicals include substituted or unsubstituted alkylidene, substituted or unsubstituted alkylene, amino tri(alkylene) having at least n+1 carbon atoms, amino di(alkylene) having at least n+1 carbon atoms, alkylenediamine tetra(alkylene) or a dialkylenetriamine penta(alkylene) radical. In each case, the alkylene group in parenthesis is connected to a phosphonic acid group. Preferably, all alkylene groups independently have 1 to 4 carbon atoms.

Exemplary compounds in which the Z group is an amino tri(alkylene) radical includes amino tri(ethylidene phosphonic acid), amino tri(isopropylidene phosphonic acid), amino di(methylene phosphonic acid) mono(isopropylidene phosphonic acid), and amino mono(methylene phosphonic acid) di(ethylidene phosphonic acid). Exemplary compounds in which the Z group is a substituted or unsubstituted alkylidene radical includes methylene diphosphonic acid, ethylidine diphosphonic acid, 1-hydroxy propylidene diphosphonic acid. Exemplary compounds in which the Z group is an alkylenediaminetetra(alkylene) or a dialkylenetriamine penta(alkylene) radical include hexamethylenediaminetetra(methylene phosphonic acid) and diethylenetriaminepenta(methylenephosphonic acid).

In one embodiment, the phosphonic acid, or its physiologically compatible salt, is represented by the following formula:

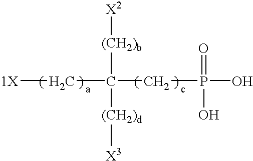

wherein each of a, b, c, and d are independently selected from integers from 0 to 4, preferably 0 or 1; X¹ is a phosphonic acid group (i.e., P(OH)₂O), hydroxy, amine or hydrogen; and X² and X³ are independently selected from the group consisting of halogen, hydroxy, amine, carboxy, alkylcarbonyl, alkoxycarbonyl, or substituted or unsubstituted phenyl, and methyl. Exemplary substituents on the phenyl are halogen, hydroxy, amine, carboxy and/or alkyl groups. A particularly preferred species is that wherein a, b, c, and d in are zero, specifically the tetrasodium salt of 1-hydroxyethylidene-1,1-diphosphonic acid, also referred to as tetrasodium etidronate, commercially available from Monsanto Company as DeQuest® 2016 diphosphonic acid sodium salt or phosphonate.

Aqueous solutions comprising the following components have been found to be particularly useful in disinfecting contact lenses that contain silver nanoparticles, or for non-silver containing, soft contact lenses that are cleaned or stored in contact lens cases containing silver nanoparticles: PHMB less than 1 ppm; dexpanthenol 0.01% to 1%; tyloxapol 0.01% to 1%; Na₂HPO₄ less than 0.06%; EDTA less than 0.2%; poloxamer 0.01% to 1%; PVP 0.01% to 1%; sorbitol at least 1%; and chloride ion less than 1000 ppm. Preferred are those solutions having the following components: PHMB less than 0.5 ppm; dexpanthenol 0.01% to 0.1%; tyloxapol 0% to 0.5%; Na₂HPO₄0.001% to 0.05%; EDTA 0.003% to 0.005%; poloxamer 0.001% to 0.5%; PVP 0.1% to 0.3%; sorbitol at least 4%; and chloride ion less than 500 ppm

Aqueous solutions that contain less than 0.5 ppm PHMB and can obtain at least a 1 log reduction in C. albicans within 15 minutes of contact with the lens are of particular interest. Also, of interest are those solutions that contain less than 0.055 ppm PHMB and can obtain at least 1.0, or 1.5 log, reduction in C. albicans within 15 minutes, or at least a 2.0 log reduction in C. albicans within 30 minutes.

The contact lens solutions described herein are used for disinfecting or packaging soft contact lenses that contain silver, and particularly silver nanoparticles, or soft contact lenses that are disinfected or stored in contact lens cases containing silver, and particularly silver nanoparticles. Such methods comprise contacting the contact lens with the compositions at conditions effective to provide the desired treatment to the contact lens. Contacting at or about ambient temperature is very convenient and useful. The contacting preferably occurs for a time in the range of about 5 minutes or about 1 hour to about 12 hours or more.

The contact lens can be contacted with the lens care solutions by immersing the lens in the solutions. During at least a portion of the contacting, the lens container holding the contact lens can be agitated, for example, by shaking the container to at least facilitate removal of deposit material from the lens. After such contacting step, the contact lens can be manually rubbed to remove further deposit material from the lens. The cleaning method can also include rinsing the lens substantially free of the lens care solutions prior to placing the lens on the eye.

The lens care solutions can be used with all categories of contact lenses such as hard, soft, rigid and soft gas permeable, and silicone (including both hydrogel and non-hydrogel) lenses. The compositions, however, are particularly formulated for uses with soft lenses, including soft silicone lenses. Many of these soft lenses, especially those formulated for extended wear, are presently prepared from high-Dₖ silicone-containing materials.

The lens care solutions are particularly formulated as a multi-purpose solution. The multi-purpose solutions will typically include one or more antimicrobial components in sufficient concentrations to destroy harmful microorganisms on the surface of a contact lens within the recommended minimum soaking time. The recommended minimum soaking time is included in the package instructions for use of the lens care compositions.

The lens solutions can also be formulated as a preserving solution, a cleaning solution or as a storage solution for contact lenses. One of ordinary skill in the art would know how to adjust the formulation for each of these respective applications. The lens care solutions in combination with its container or bottle and packaging, including instructions for use in accordance with a specified regimen, provides an improved kit, package, or system for the care of contact lenses.

The following non-limiting examples illustrate certain aspects of the present invention.

### EXAMPLES

Each of the stated formulation components are reported as % w/v unless noted as ppm.

### Example 1

A solution is prepared by blending the following components: PHMB 0.5 ppm; bis-TRIS-propane 0.1%; dexpanthenol 0.02%; sorbitol 4% ; Pluronic F-127 0.1%; and tyloxapol 0.02%. The pH is adjusted to near neutral with phosphoric acid.

### Example 2

A solution is prepared by blending the following components: PHMB 0.055 ppm; bis-TRIS-propane 0.1%; dexpanthenol 0.02%; sorbitol 5%; Pluronic F-127 0.1%; and tyloxapol 0.02%. The pH is adjusted to near neutral with phosphoric acid.

### Example 3

A solution is prepared by blending the following components: PHMB 0.055 ppm; tromethamine 0.1%; dexpanthenol 0.02%; sorbitol 5%; Pluronic F-127 0.1%; and tyloxapol 0.02%. The pH is adjusted to near neutral with boric acid.

### Example 4

A solution is prepared by blending the following components: PHMB 0.055 ppm; tromethamine 0.1%; dexpanthenol 0.02%; sorbitol 5%; Pluronic F-127 0.1%; and tyloxapol 0.02%. The pH is adjusted to near neutral with phosphoric acid.

### Example 5

A solution is prepared by blending the following components: PHMB 1.0 ppm bis-TRIS-propane 0.1% dexpanthenol 1.0% sorbitol 4% Pluronic F -127 0.1% Chemophore RH40 0.1%. The pH is adjusted to near neutral with phosphoric acid.

### Example 6

A solution is prepared by blending the following components: PHMB 1.0 ppm; bis-TRIS-propane 0.1%; dexpanthenol 0.2%; sorbitol 4%; Pluronic F-127 0.1%; and tyloxapol 0.02%. The pH is adjusted to near neutral with phosphoric acid.

### Example 7

A solution is prepared by blending the following components: PHMB 1.0 ppm; bis-TRIS-propane 0.1%; dexpanthenol 0.1%; sorbitol 4%; Pluronic F-127 0.1%; and Chemophore RH40 0.1%. The pH is adjusted to near neutral with phosphoric acid.

### Examples 8

A solution is prepared by blending the following components: PHMB 0.055 ppm; dexpanthenol 0.02%; tyloxapol 0.02%; Na₂HPO₄0.01%; Pluronic 17R4 0.05%; and PVP (K-90) 0.4%. The pH is adjusted to near neutral with phosphoric acid and/or sodium hydroxide.

### Examples 9

A solution is prepared by blending the following components: PHMB 0.055 ppm; dexpanthenol 0.02%; tyloxapol 0.02%; Na₂HPO₄0.001%; Pluronic F-68NF 0.1%; and Sorbitol 5%. The pH is adjusted to near neutral with phosphoric acid.

### Example 10

A solution is prepared by blending the following components: PHMB 0.055 ppm; dexpanthenol 0.2%; Pluronic F-127 0.1%; Na₂HPO₄0.001%; tyloxapol 0.02%; sorbitol 5%; and EDTA 0.004%. The pH is adjusted to near neutral with phosphoric acid and/or sodium hydroxide.

### Example 11

A solution is prepared by blending the following components: PHMB 0.055 ppm; dexpanthenol 0.2%; Pluronic F-127 0.1%; Na₂HPO₄0.001%; tyloxapol 0.02%; sorbitol 2%; and propylene glycol 1%. The pH is adjusted to near neutral with phosphoric acid and/or sodium hydroxide.

### Example 12

A solution is prepared by blending the following components: PHMB 0.055 ppm; dexpanthenol 0.2%; Pluronic F-127 0.1%; Na₂HPO₄0.001%; tyloxapol 0.02%; sorbitol 5%; and PVP (K-90) 0.5%. The pH is adjusted to near neutral with phosphoric acid and/or sodium hydroxide.

### Example 13

A solution is prepared by blending the following components: PHMB 0.055 ppm; dexpanthenol 0.2%; Pluronic F-127 0.1%; Na₂HPO₄0.001%; tyloxapol 0.02%; sorbitol 5%; and Pluronic 17R4 0.07%. The pH is adjusted to near neutral with phosphoric acid and/or sodium hydroxide.

### Example 14

A solution is prepared by blending the following components: PHMB 0.055 ppm; dexpanthenol 0.2%; Na₂HPO₄0.001%; tyloxapol 0.02%; sorbitol 5%; and Pluronic F-68LF 0.1%; The pH is adjusted to near neutral with phosphoric acid and/or sodium hydroxide.

### Example 15

A solution is prepared by blending the following components: PHMB 1.0 ppm; dexpanthenol 0.02%; sodium borate 0.09%; boric acid 0.85%, Tetronic 1107 0.5%; and NaCl 0.001%. The pH is adjusted to near neutral with phosphoric acid and/or sodium hydroxide.

### Example 16

A solution is prepared by blending the following components: PHMB 1.0 ppm; dexpanthenol 0.02%; Dequest 2016 0.1%; Na₂HPO₄0.1%; boric acid 0.058%, Tetronic 1107 0.5%; Pluronic F-127 0.5%; and glycerin 0.5%. The pH is adjusted to near neutral with phosphoric acid and/or sodium hydroxide.

### Example 17

A solution is prepared by blending the following components: PHMB 1.0 ppm; dexpanthenol 0.02%; Dequest 2016 0.1%; Na₂HPO₄0.31%; boric acid 0.85%, Tetronic 1107 0.5%; and Pluronic F-127 0.5%. The pH is adjusted to near neutral with phosphoric acid and/or sodium hydroxide.

### Example 18

A solution is prepared by blending the following components: Polyquarternium-1 10.0 ppm; dexpanthenol 0.02%; Dequest 2016 0.1%; Na₂HPO₄0.1%; boric acid 0.058%, Tetronic 1107 0.5%; Pluronic F-127 0.5%; sorbitol 1.0% and Na₂CO₃0.1%. The pH is adjusted to near neutral with phosphoric acid and/or sodium hydroxide.

### Example 19

A solution is prepared by blending the following components: PHMB 1.0 ppm; dexpanthenol 0.02%; Dequest 2016 0.1%; Na₂HPO₄0.1%; boric acid 0.058%, Tetronic 1107 0.5%; Pluronic F-127 0.5%; sorbitol 1.0% and Na₂CO₃0.1%. The pH is adjusted to near neutral with phosphoric acid and/or sodium hydroxide.

### Example 20

A solution is prepared by blending the following components: alexidine 1.0 ppm; dexpanthenol 0.02%; Dequest 2016 0.1%; Na₂HPO₄0.1%; boric acid 0.058%, Tetronic 1107 0.5%; Pluronic F-127 0.5%; and glycerin 0.5%. The pH is adjusted to near neutral with phosphoric acid and/or sodium hydroxide.

### Example 21

A solution is prepared by blending the following components: Polyquarternium-1 10 ppm; dexpanthenol 0.02%; Dequest 2016 0.1%; Na₂HPO₄0.1%; boric acid 0.058%, Tetronic 1107 0.5%; Pluronic F-127 0.5%; and glycerin 0.5%. The pH is adjusted to near neutral with phosphoric acid and/or sodium hydroxide.

### Example 22

A solution is prepared by blending the following components: PHMB 1.0 ppm; dexpanthenol 0.02%; Dequest 2016 0.1%; Na₂HPO₄0.1%; boric acid 0.058%, Tetronic 1107 0.5%; Pluronic F-127 0.5%; and glycerin 0.5%. The pH is adjusted to near neutral with phosphoric acid and/or sodium hydroxide.

### Example 23

A solution is prepared by blending the following components: PHMB 1.0 ppm; dexpanthenol 0.02%; Dequest 2016 0.1%; Na₂HPO₄0.1%; boric acid 0.058%, PVP C30 0.5%; PVP K90 1.0%; and glycerin 0.5%. The pH is adjusted to near neutral with phosphoric acid and/or sodium hydroxide.

### Example 24

A solution is prepared by blending the following components: PHMB 1.0 ppm; dexpanthenol 0.02%; Dequest 2016 0.1%; Na₂HPO₄0.1%; boric acid 0.058%, CMC 0.5%; PVP K90 1.0%; and glycerin 0.5%. The pH is adjusted to near neutral with phosphoric acid and/or sodium hydroxide.

### Example 25

A solution is prepared by blending the following components: Polyquartemium-1 10 ppm; dexpanthenol 0.02%; EDTA 0.1%; Na₂HPO₄0.1%; boric acid 0.058%, CMC 0.5%; PVP K90 1.0%; and xylose 0.5%. The pH is adjusted to near neutral with phosphoric acid and/or sodium hydroxide.

### Example 26

A solution is prepared by blending the following components: alexidine 1.0 ppm; dexpanthenol 0.02%; EDTA 0.1%; Na₂HPO₄0.1%; boric acid 0.058%, CMC 0.5%; PVP K90 1.0%; and trahalose 0.5%. The pH is adjusted to near neutral with phosphoric acid and/or sodium hydroxide.

### Example 27

A solution is prepared by blending the following components: PHMB 1.0 ppm; dexpanthenol 0.02%; Dequest 2016 0.1%; TRIS 0.1%; boric acid 0.058%, Tetronic 908 0.5%; PVP K90 0.5%; and tyloxapol 0.05%. The pH is adjusted to near neutral with phosphoric acid and/or sodium hydroxide.

### Example 28

A solution is prepared by blending the following components: PHMB 1.0 ppm; dexpanthenol 0.02%; Dequest 2016 0.1%; Tetronic 1107 0.5%; CMC 0.5%; glycerin 1.0% and mannitol 2.0%. The pH is adjusted to near neutral with phosphoric acid and/or sodium hydroxide.

### Example 29

A solution is prepared by blending the following components: PHMB-CG* 1.0 ppm; dexpanthenol 0.02%; Dequest 2016 0.1%; Tetronic 1107 0.5%; CMC 0.5%; glycerin 1.0% and mannitol 2.0%. The pH is adjusted to near neutral with phosphoric acid and/or sodium hydroxide.

### Example 30

A solution is prepared by blending the following components: PHMB 1.0 ppm; Polyquarternium-1 5.0 ppm; dexpanthenol 0.02%; Dequest 2016 0.1%; Tetronic 1107 0.5%; CMC 0.5%; glycerin 1.0% and mannitol 2.0%. The pH is adjusted to near neutral with phosphoric acid and/or sodium hydroxide.

### Example 31

A solution is prepared by blending the following components: PHMB 0.5 ppm; alexidine 0.5 ppm; dexpanthenol 0.02%; Dequest 2016 0.1%; Tetronic 1107 0.5%; CMC 0.5%; and mannitol 5.0%. The pH is adjusted to near neutral with phosphoric acid and/or sodium hydroxide.

### Example 32

A solution is prepared by blending the following components: PHMB 0.5 ppm; alexidine 0.5 ppm; dexpanthenol 0.02%; Dequest 2016 0.1%; TRIS 0.1%; Na₂HPO₄0.1%; Tetronic 1107 0.5%; CMC 0.5%; and mannitol 2.5%. The pH is adjusted to near neutral with phosphoric acid and/or sodium hydroxide.

### Example 33

PHMB (Polyamino propyl biguanide hydrochloride, 2 g, 0.0011 mole,) and HMBDA (1,6-bis(cyanoguanadino)hexane, 0.3 g, 0.0012 mole are mixed and ground together, and placed in a 100 mL round bottom flask. Concentrated Hydrochloric acid (100 μL) is then added to the PHMB/HMBDA. The mixture is slowly heated to 100° C. until all the liquid is driven off. The heat is then increased to 150° C. to 160° C. and held for 4 hours. The reaction mixture is cooled to room temperature providing 1.32 g of crystalline material, PHMB-CG*.

We claim:

1. A method of disinfecting or cleaning a contact lens, the method comprising:
positioning a contact lens comprising silver in a lens case or lens package; and
contacting the positioned contact lens with a contact lens solution, wherein the lens solution comprises less than 0.05% w/v chloride ion.

2. The method of claim 1 wherein the silver in the contact lens comprises primarily silver nanoparticles.

3. The method of claim 1 wherein the lens solution comprises less than 0.01% w/v chloride ion.

4. The method of claim 1 wherein the lens solution comprises less than 0.005% w/v chloride ion.

5. The method of claim 1 wherein the lens solution further comprises a cationic antimicrobial component selected from the group consisting of poly(hexamethylene biguanide) or PHMB-CG*, which is present from 0.01 ppm to 3 ppm, α-[4-tris(2-hydroxyethyl)ammonium chloride-2-butenyl]poly[1-dimethylammonium chloride-2-butenyl]-ω-tris(2-hydroxyethyl)ammonium chloride, which is present from 1 ppm to 100 ppm, and any mixture thereof.

6. The method of claim 1 wherein the lens solution further comprises dexpanthenol, sorbitol or any mixture thereof.

7. The method of claim 1 wherein the contact lens is a soft, silicon lens.

8. A sealed, contact lens package comprising a contact lens that comprises silver, and a contact lens solution comprising less than 0.05% w/v chloride ion.

9. The lens package of claim 8 wherein the lens solution comprises less than 0.01% w/v chloride ion.

10. The lens package of claim 8 wherein the lens solution comprises less than 0.005% w/v chloride ion.

11. The lens package of claim 8 wherein the silver in the contact lens comprises primarily silver nanoparticles.

12. The lens package of claim 8 wherein the contact lens is a soft, silicon lens.

13. A method of disinfecting or cleaning a contact lens, the method comprising
positioning the contact lens in a lens case that comprises silver; and
contacting the positioned contact lens with a contact lens solution, wherein the lens solution comprises less than 0.05% w/v chloride ion.

14. The method of claim 13 wherein the lens solution comprises less than 0.01% w/v chloride ion.

15. The method of claim 13 wherein the lens solution comprises less than 0.005% w/v chloride ion.

16. The method of claim 13 wherein the silver in the lens case comprises primarily silver nanoparticles.

17. The method of claim 13 wherein the lens solution further comprises a cationic antimicrobial component selected from the group consisting of poly[dimethylimino-2-butene-1,4-diyl]chloride, α-[4-tris(2-hydroxyethyl)ammonium chloride-2-butenylpoly[1-dimethylammonium chloride-2-butenyl]-ω-tris(2-hydroxyethyl) ammonium chloride, myristamidopropyl dimethylamine, benzalkonium halides, alexidine and salts thereof, hexamethylene biguanides and salts thereof and their polymers, and mixtures thereof.

18. The method of claim 13 wherein the contact lens is a soft, silicon lens.
